(19) United States
(12) Patent Application Publication
(10) Pub. No.: US 2024/0226532 A9
(43) Pub. Date: Jul. 11, 2024

(54) POWER CABLE SYSTEM FOR MEDICALLY IMPLANTED DEVICES

(71) Applicant: Jarvik Heart Inc., New York, NY (US)

(72) Inventors: Robert Jarvik, New York, NY (US); Peter W.J. Hinchliffe, West Yarmouth, MA (US)

(73) Assignee: Jarvik Heart Inc., New York, NY (US)

(21) Appl. No.: 18/548,633

(22) PCT Filed: Mar. 9, 2022
(86) PCT No.: PCT/US2022/019438

§ 371 Date: Sep. 1, 2023

Related U.S. Application Data
(60) Provisional application No. 63/160,472, filed on Mar. 12, 2021.

(51) Int. Cl. A61M 60/178; A61M 60/88

(52) U.S. Cl. CPC A61M 60/178; A61M 60/88; A61M 2210/0687

(57) ABSTRACT

A cable system for providing power to an implantable device implanted in a patient having a pedestal base mountable to a skull of a patient. An internal cable extends from the pedestal base and is electrically connectable to the implantable device. An external cable is connectable to the post of the pedestal base. A locking ring is releasably mountable to the post and is movable from a first configuration to a second configuration, wherein in the first configuration the locking ring locks to the post to secure the external cable to the pedestal base and in the second configuration the locking ring releases from the post to release the external cable from the pedestal base.

This application claims priority to provisional application 63/160,472 filed on Mar. 12, 2021, the entire contents of which are incorporated herein by reference.

### BACKGROUND OF THE INVENTION

### Technical Field

This application relates to a cable system for powering implantable medical devices.

### Background of Related Art

Many medical devices which utilize electric power have been developed for long term implantation. If the power required is low enough, such as with pacemakers, and various types of muscle and nerve stimulators, implanted batteries can reliably store enough energy for years of operation. These may utilize tiny amounts of power measured in milliamps using intermittent brief bursts of stimulation. Devices such as blood pumps, heart assist devices or total artificial hearts operate continuously and require thousands of times as much energy as pacemakers. A battery which powers a pacemaker for a decade would power an artificial heart for less than an hour.

Percutaneous leads are means of accessing the tissues beneath the skin. Many types of percutaneous leads have been developed and include catheters for fluid access, fabric-covered pneumatic tubes, and electric cables with large subcutaneous flanges for soft tissue ingrowth to fix the device in place and provide a barrier to bacterial infections.

A major cause of infection of percutaneous leads is trauma to the tissues where the device penetrates the skin. Motion of the tube or cable relative to the skin tears the cellular junction of the body tissue to the prosthetic material. This occurs repeatedly and prevents tight healing and permits bacteria to enter.

The most successful type of percutaneous lead uses rigid fixation to bone to prevent motion of the device and places the device in a position where virtually no motion of the skin over the bone occurs. This protects the junction of the skin and percutaneous lead from trauma. Skull-mounted devices of this type have proven highly effective. In addition to excellent stabilization on the skull, the tissues of the scalp are highly vascular and adapted to resist wound infection, as an evolutionary mechanism to protect the brain.

U.S. Pat. No. 5,904,646 discloses a cable system for providing power to an artificial heart or assist device located in the chest via a skull-mounting position. The wires are tunneled through the tissues of the neck and across the chest wall to reach the heart, and are designed to withstand a great deal of flexing and torsional strain as the patient bends and turns the head and neck. A serpentine cable having several zig-zag loops lying along the neck in order to relieve strain on the cables is implanted utilizing several small incisions. The power cable and connector part are separable from the post of the large diameter flange of the pedestal attached to the skull. The cable is attached to the flange after tunneling to permit zig-zag tunneling with minimal trauma. A keyed configuration of the pedestal and cable connector is provided which permits the plugging and unplugging of the connector in the back or side of the head.

Although the cable systems of U.S. Pat. No. 5,904,646 are effective and advantageous over preceding and current cable systems, improvements to the securement of the pedestal and attachment of the cable system would be beneficial.

### SUMMARY OF THE INVENTION

The present invention provides improved cable systems for implantable medical devices such as blood pumps. The cable system of the present invention enhances attachment of the pedestal to the skull and/or facilitates attachment and replacement of the cable to the pedestal. Various features to achieve these advantages/enhancements are described in detail below.

In accordance with one aspect of the present invention, a cable system for providing power to an implantable device implanted in a patient is provided comprising:
a. a pedestal base mountable to a skull of a patient, the pedestal base including a post extending outwardly therefrom;
b. an internal cable electrically connectable to the implanted device and pedestal base; and
c. an external cable connectable to the post of the pedestal base, the external cable having a locking ring releasably mountable to the post, the locking ring movable from a first configuration to a second configuration, wherein in the first configuration the locking ring locks to the post to secure the external cable to the pedestal base and in the second configuration the locking ring releases from the post to release the external cable from the pedestal base.

In some embodiments, the internal cable is passed through the patient prior to inserting the internal cable through the post and prior to mounting the pedestal base to the skull.

In some embodiments, the first configuration of the locking ring is substantially oval and the second configuration is substantially circular. Other configurations are also contemplated.

In some embodiments, the locking ring is non-removably attached to the external cable; in other embodiments it is removably attached. The locking ring, in some embodiments, can have an irregular surface to facilitate manual squeezing of the locking ring from the first configuration to the second configuration.

In some embodiments, the locking ring has a first end, a second end and an engagement member extending from the second end, wherein the engagement member extends inwardly toward a longitudinal axis of the locking ring. In some embodiments, the engagement member engages an inner surface of the post of the pedestal base.

In some embodiments, the locking ring has a circumferential wall between a first and second end, and the circumferential wall can include an inwardly extending structure permanently attaching the locking ring to a cylindrical section of the external cable. The inwardly extending structure can comprise crimps or barbs.

In some embodiments, the external cable has a cylindrical section with an asymmetric cutout to mate with an asymmetric head of the post of the pedestal base, and a connector is positioned within the cutout which is attachable to a connector within a cutout in the post.

In some embodiments, the pedestal base has a first opening and a series of screw receiving openings, the screw receiving openings each dimensioned to receive a screw for attachment of the pedestal base to the skull, and the first opening is configured to receive bone chips for tissue ingrowth. In some embodiments, the first opening is dimensioned so as not enable insertion or securement of a bone screw.

A coating can be provided in some embodiments on at least a portion of the pedestal base to enhance tissue ingrowth.

In accordance with another aspect of the present invention, a cable system for providing power to an implantable device implanted in a patient is provided, the cable system comprising:
a) a pedestal base mountable to a skull of a patient, the pedestal base including a post extending outwardly therefrom, the pedestal base further having an upper surface and a lower surface and a first and second openings extending from the upper surface to the lower surface;
b) an internal cable extending from the pedestal base and electrically connectable to the implanted device; and
c) a first screw extending through the first opening and a second screw extending through the second opening, wherein the first and second screws have an initial captured position extending through the first and second openings so distal ends of the first and second screws extend beyond (distal) the lower surface of the pedestal base;
d) wherein the first and second screws are in the initial captured position during shipping and are moved to a second position wherein a distal tip of the first and second screws is flush with or proximal of the lower surface so as not to extend beyond the lower surface for mounting of the pedestal base to the skull.

In some embodiments, the bone screws are self-tapping screws.

In some embodiments, the internal cable is passed through the patient prior to inserting the internal cable through the post and prior to mounting the pedestal base to the skull.

The system can include an external cable connectable to the post of the pedestal base.

In some embodiments, the cable system has a locking ring releasably mountable to the post, the locking ring movable from a first configuration to a second configuration, wherein in the first configuration the locking ring locks to the post to secure the external cable to the base and in the second configuration the locking ring releases from the post to release the external cable from the pedestal base.

In some embodiments, portions of the pedestal base which come into contact with tissue are coated to provide tissue ingrowth.

In some embodiments, in the first configuration the locking ring is substantially oval and in the second configuration is substantially circular. Other configurations are also contemplated. The locking ring can have in some embodiments a circumferential wall between a first and second edge or end, and the circumferential wall can include an inwardly extending structure permanently attaching the locking ring to a cylindrical section of the internal cable.

In accordance with another aspect of the present invention, a method for providing power to an implantable device implanted in a patient is provided comprising:
a. mounting a pedestal base to skull of a patient via application of a set of screws, the pedestal base having a post and receiving an internal cable extending from the implantable device and tunneled from inside of the chest through the neck and to the skull; and
b. attaching an external cable to the post of the pedestal base, wherein the external cable is attached by altering a shape of a locking ring to position the locking ring over the post and releasing the locking ring to return to its initial shape to secure to the post.

In some embodiments, the locking ring is releasable from the post by altering the initial shape of substantially oval to a substantially round shape.

In some embodiments, the set of screws are self-tapping screws and the step of mounting the pedestal base comprises applying self tapping screws through openings in the pedestal base to engage the skull.

In some embodiments, the screws have an initial captured position extending through the openings in the pedestal base so the distal tip of the screws extend beyond a lower surface of the base and prior to mounting the pedestal to the skull, the screws are retracted from their captured position to a second position wherein distal tips of the screws do not extend beyond (past) the lower surface of the pedestal base, e.g., are flush with or proximal of the lower surface, for mounting of the pedestal base to the skull.

### BRIEF DESCRIPTION OF THE DRAWINGS

So that those having ordinary skill in the art to which the subject invention appertains will more readily understand how to make and use the surgical systems disclosed herein, preferred embodiments thereof will be described in detail hereinbelow with reference to the drawings, wherein:

FIG. 1 is an illustration of a patient viewed from the back showing the skull mounted post, zig-zag wires across the neck and blood pump;

FIG. 2 is an exploded view of the cable system in accordance with one embodiment of the present invention;

FIG. 2A is a side view of the locking ring of FIG. 2 showing the taper; and

FIG. 3 is a cross-sectional view showing for illustrative purposes one bone screw in the distal shipping position and one bone screw in the retracted ready-for-insertion position.

### DETAILED DESCRIPTION

The cable system of U.S. Pat. No. 5,904,646 (hereinafter the '646 patent) has been used successfully used for powering long term implanted devices. However, the inventors recognized that there is room for improvement of these cable systems in one or more of the following ways: 1) enhance pedestal attachment and securement to the skull; 2) reduce the number of steps in the procedure; 3) facilitate cable attachment and replacement; and/or 4) reduce trauma during cable replacement. These improvements of the present invention are implemented by one or more of the following new features: a) a plate (pedestal) with captured screws; b) self-tapping screws for attachment to the skull; c) microsphere coating on select parts of the pedestal base to enhance tissue ingrowth; and d) a releasable lock for removably attaching the cable to the pedestal base. Each of these features is discussed in detail below. It should be appreciated that all four features a, b, c and d can be implemented in the cable system of the present invention, or alternatively, only one, only two or only three of the features could be implemented in the cable system of the present invention. It should also be appreciated that all four above listed improvements 1, 2, 3 and 4 can be achieved in the cable system of the present invention, or alternatively, only one, only two or only three of the improvements could be implemented in the cable system of the present invention depending on the features a-d utilized.

As used herein, the term “proximal” denotes portions or sections closer to the user and the term “distal” denotes portions or sections further from the user.

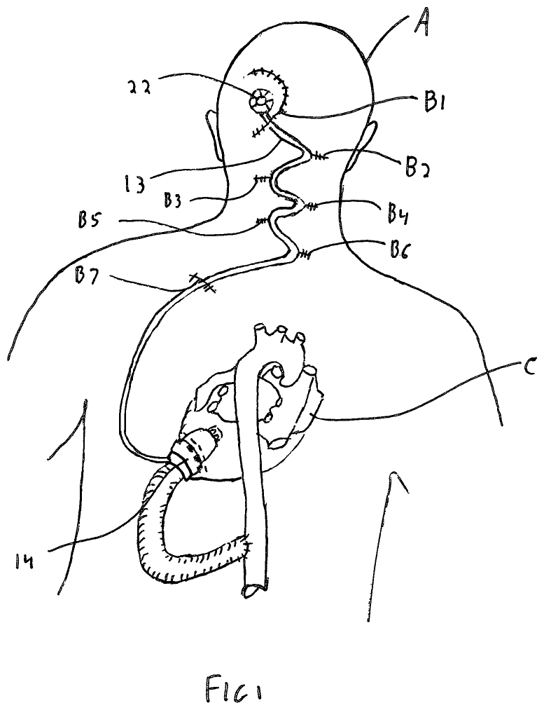

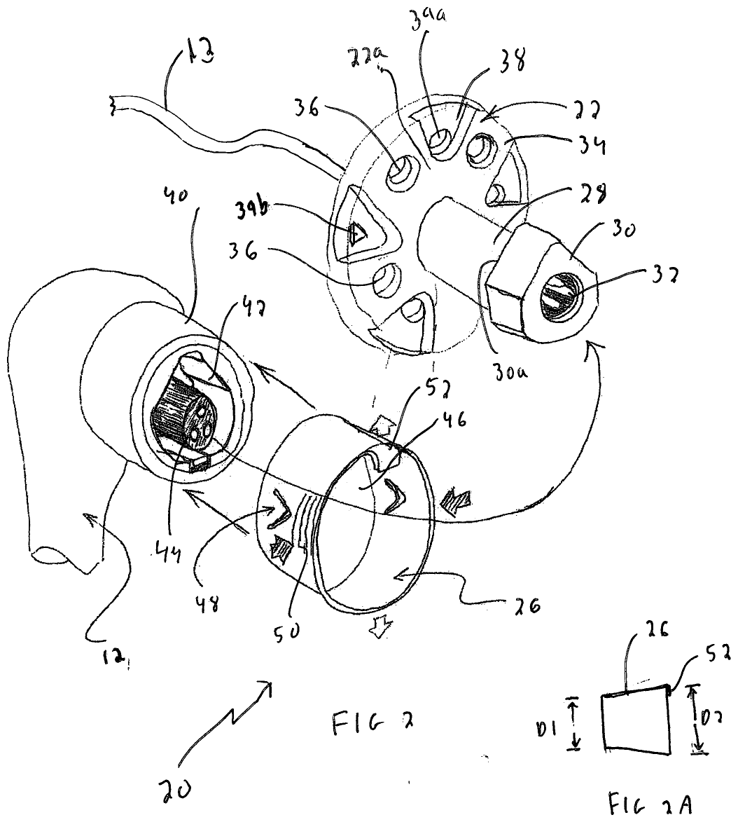

With reference to FIGS. 1 and 2, pedestal base 22 is mounted to the skull A by bone screws in position on the side and back of the head behind the ear. A relatively large incision B1 is used to create a pocket into which the flange 34 of pedestal base 22 is inserted and attached to the skull with screws. A series of small skin incisions B2-B6 are placed in staggered fashion down the neck and are used to implant the internal cable 13 so that it assumes a serpentine curvature as it lies in the subcutaneous tissues of the neck. The internal cable 13 is connected to an intraventricular blood pump 14 which is placed within the heart C and connected to a control system outside the body.

The external cable 12 is connected to an external power source and terminates at one end at a connector 44. The connector 44 is inserted through, and can be removably attached, to the post 28 of skull-mounted pedestal base 22.

The post 28 extends from flange 34 of pedestal base 22. A series of openings 36 extend in a circular array through the flange 34, although other arrays are also contemplated. Openings 36 are configured and dimensioned for receiving bone screws as described below for mounting the pedestal 22 to the skull. Cutouts (recesses) 38, which are illustratively substantially triangular in configuration, have holes for bone chips. The holes can be circular in shape as shown by hole 39a. Alternatively, the holes can be substantially triangular shaped, shown by hole 39b, or other non-circular shape, so that a bone screw cannot fit therethrough, thus not enabling insertion of the screw. Alternatively, holes of circular or other shape which are larger than the screw head could be utilized which would not be able to hold (secure) a screw. Such shaped holes which would not enable insertion and/or securement would therefore differentiate for the user the holes designed for the screws from the holes designed for bone. The recesses 38 and holes 39a, 39b provide room for placement/packing of bone chips for tissue ingrowth. A different number of bone receiving holes and/or screw receiving holes than those illustrated in FIG. 2 could be provided as well as a different arrangement of the holes and/or different configuration.

The post 28 has an asymmetric substantially triangular or mushroom shaped head 30 with an opening 32 for receipt therein of the connector 44 of the external cable 12. The other end of the post 28 is attached to, or integral with, the flange 34 of pedestal base 22 and extends away from the upper (proximal) surface 22a of flange 34. Other asymmetric shaped heads are also contemplated.

External cable 12 has a cylindrical section 40 with a cutout 42 formed of a shape to mate with the asymmetric head 30 of post 28 of pedestal base 22. Connector 44 is positioned within the cutout 42. Locking ring 26 (also referred to herein as the locking sleeve or lock) is positioned over the cylindrical section 40. The locking ring 26 is shown separated from the cylindrical section 40 in FIG. 2 for illustrative purposes but in preferred embodiments would be non-removably attached to the cylindrical section 40 in manufacture. In some embodiments, the locking ring 26 is in the form of a spring steel ring, spring loaded to a locking (oval) position. The locking ring 26 connects to the head 30 of the post 28 of pedestal base 22.

In the cable system disclosed in the '646 patent, the pedestal cable is frictionally fit over the pedestal base and has tight tolerance fit requirements over the pedestal base and can become loose and come apart over time. Further, the close tolerances of the press fit can create connection challenges if the cables are made by different manufacturers. The locking sleeve (ring) of the present invention reduces the likelihood of loosening. It provides a positive lock and tactile and/or audible click attachment. The locking ring 26 is placed over the head 30 of pedestal post 28 and is retained on the head 30 by lip 52 which forms an engagement member (structure) at a distal end (or edge) of the ring 26 extending inwardly toward the longitudinal axis of the locking ring 26. That is, the lip 52 of lock 26 overhangs to catch the head 30 of post 28, i.e., engage an inner (distal) surface of the head 30.

The lock 26 in preferred embodiments is ovalized and has a taper in a direction toward the cable 12 so that diameter D1 on the cable side is less than the diameter D2 on the pedestal side (see FIG. 2A). Lock 26 can include crimps or barbs 48 or other structure extending inwardly from a circumferential wall to permanently attach the locking ring 26 to cylindrical section 40 of external cable 12. An irregular surface such as a knurled or textured surface 50 can facilitate gripping for the squeeze release of lip 52 (and thus ring 26 and attached cable 12) from head 30. That is, to release the cable 12 from the pedestal base 22, the locking ring 26 is squeezed from its initial oval shape (configuration) to create a more circular shape (configuration). This releases the lip 52 from engagement with the inner surface 30a of head 30 to enable release of the cable 12. The locking ring pedestal/cable connection of the present invention reduces the force applied to the skull when the cable is attached as well as removed as compared to the frictional fit of the '646 patent since as much as 5 lbs. of force could be required to attach or remove the cable in the '646 patent, with the patient's head being held as a counterforce. Also, due to the tight tolerances of the frictional fit of the connector of the '646 patent, it is sometimes hard to push the cable into the pedestal. The cable connection of the present invention thereby improves ease of use, reduces connection mistakes, reduces the force required for release (pushing the skull in the opposing direction) and provides a retrofittable snap in design.

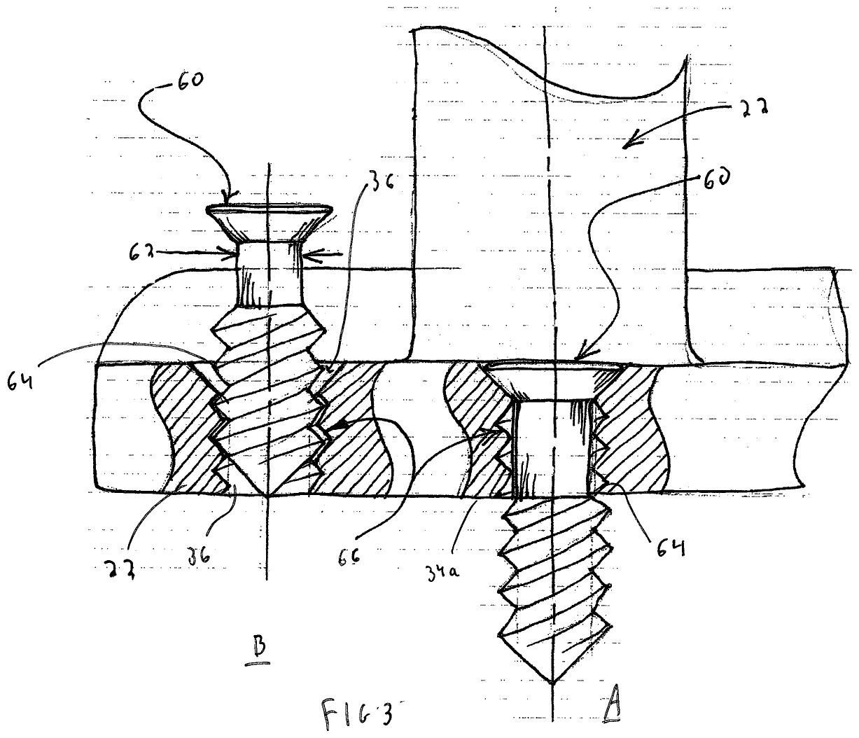

Turning to the bone screw features a and b listed above, and with reference to FIG. 3, two of the multiple bone screws are shown to explain the features of the invention. Multiple bone screws would be utilized in clinical use, extending through the openings 36 in the flange 34 of pedestal base 22.

The openings 36 (and thus the screws) can be arranged in a circular array as shown or alternatively arranged in other arrays. The pedestal base 22 is shown having a circular configuration, but other configurations are also contemplated, and thus the openings 36 could be arranged to correspond to the non-circular shape of the base 22. The pedestal base 22 can also be shaped to be triangular or otherwise have a “vertex” or other shape to provide an arrow-like indicator to facilitate orientation of the pedestal base 22 by the user. Other orientation markers are also contemplated. In some embodiments, the shape of the pedestal base 22 could be similar to head 30 of post 28 to provide aligned vertices for orientation so the arrow-like indicator would be pointing in the correct orientation for implantation which in turn will provide a more streamlined/straighter cable connection to the pedestal base 22.

Bone screws 60 in some embodiments can be self-tapping screws. Bone screw 60 in the illustrated embodiment is captured in pedestal base 22. In the shipping condition, bone screw 60 is in position “A” where it extends fully through the threaded portion 66 of the threaded hole 36 in the pedestal base 22 such that its distal portion, e.g., at least its distal tip and at least its distal threads, extends below (beyond/distal) the lower surface (undersurface) 34a of the pedestal base 22. Holes 36 are partially threaded as shown and the shank of the bone screw 60 is seated within the hole 36, with the threads of screw 60 extending past the undersurface 34a of the flange 34 of pedestal base 22. The screw 60 has an undercut 62 and a reduced shank diameter below the screw head to allow the screw 60 to rotate freely once engaged fully and held in a captured position when retracted to allow secure capture when being handled in the Operating Room and easy placement when advanced into the bone by the surgeon.

In use, for mounting of the pedestal base 22 to the skull, the screw 60 is retracted to its ready-to-insert position “B” of FIG. 3 where the distal portion of the screw is flush or above the lower surface 34a of the pedestal base 22, i.e., no portion or no substantial portion of the screw extends beyond (distal) of lower surface 34a. The screw 60 is then advanced through hole 36 into the patient's skull to position “A.” If a self-tapping screw is utilized, the self-tapping feature saves time compared to the more time-consuming step of pre-drilling and tapping holes.

Note that FIG. 3 shows one of the screws in the initial captured distal shipping position and another screw in the retracted (proximal) ready-for-insertion/mounting position for illustrative purposes. In use of preferred embodiments, each of the screws would be shipped in the distal position and then each of the screws would be retracted to place the pedestal base on the skull and then the screws advanced through the pedestal base openings into the skull.

In alternate embodiments, the pedestal is attached to the skull without the use of screws. In one embodiment, the pedestal has a mesh flange that does not require screws for attachment. This could be particularly beneficial for infants or young children who have skulls that could be too thin for screws. In another embodiment, a bio-absorbable tissue adhesive can be utilized instead of screws to hold the pedestal temporarily in place (months to years) while the skull thickness increases as the child grows, leaving a tissue scaffold under the periosteum.

In an alternate embodiment, a flat pedestal can be provided.

A microsphere or other tissue-friendly coating can be applied to portions of the pedestal which come in contact with the tissue to provide better tissue ingrowth. More specifically, the microsphere coating could be applied to the outside diameter of the post 28 in regions that engage the skin. The coating could also be applied to the pedestal base 22.

The surgical procedure for implantation of the device to achieve the configuration shown in FIG. 1 will now be described. The chest is first opened and the internal cable 13 together with the cable connector is tunneled from the inside of the chest cavity passing outward through a small incision B7 above the shoulder blade, either in back or laterally near the base of the neck. Using a tunneling instrument, the connector and cable are then sequentially passed from incision to incision (B6-B1), although more or fewer incisions than shown could be used. At each incision the connector is first brought out through the skin and then on the next step is reinserted into the same incision. After the internal cable 13 has been completely placed from the chest to the head, the connector of the internal cable 13 is inserted into the flange 34 and post 28. Then pedestal base 22, i.e., flange 34, is attached to skull by screws and the skin is closed around the flange 34 of the pedestal base 22. Next the external cable 12 is attached to pedestal 22. The locking ring 26 is squeezed to alter its shape, e.g., to a more circular configuration, so the lip 52 can pass over the head 30 of the post 28 and the connector 44 can connect to the connectors within opening 32 of head 30 of post 28. Post 28 extends from upper surface 22a of flange 34 of pedestal base 22 which is attached to the patient's skull via bone screws, e.g., the self-tapping captured bone screws described above. The locking ring 26 and underlying cylindrical section 40 of cable 12 encircle the head 30 of post 28. The locking ring 26 is then released to return the ring 26 to its normal oval configuration, wherein the lip 52 engages the inner surface 30a of head 30. This prevents the locking ring 26 from sliding off the head 30 of the post 28 and separating from the pedestal base 22. As noted above, this attachment to the post is achieved with minimal force applied to the skull. When after a period of time the cable 12 needs to be replaced, the locking ring 26 is squeezed to change its configuration to a more circular configuration to release the lip 52 of locking ring 26 from the head 30 of post 28. The locking ring 26 with attached cable 12 is then slid off the head 30 with minimal force applied to the skull. A new external cable can be attached via the locking ring 26 in the manner described above.

Although the apparatus and methods of the subject invention have been described with respect to preferred embodiments, those skilled in the art will readily appreciate that changes and modifications may be made thereto without departing from the spirit and scope of the present invention as defined by the appended claims.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the present invention, terms such as “approximately,” “generally,” “substantially,” and the like should be understood to allow for variations in any numerical range or concept with which they are associated. For example, it is intended that the use of terms such as “approximately” and “generally” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

1. A cable system for providing power to an implantable device implanted in a patient, the cable system comprising:
a. a pedestal base mountable to a skull of a patient, the pedestal base including a post extending outwardly therefrom;
b. an internal cable electrically connectable to the implantable device;
c. an external cable connectable to the post of the pedestal base, the external cable having a locking ring releasably mountable to the post, the locking ring movable from a first configuration to a second configuration, wherein in the first configuration the locking ring locks to the post to secure the external cable to the pedestal base and in the second configuration the locking ring releases from the post to release the external cable from the pedestal base.

2. The cable system of claim 1, wherein in the first configuration the locking ring is substantially oval and in the second configuration the locking ring is substantially circular.

3. The cable assembly of claim 1, wherein the locking ring is non-removably attached to the external cable.

4. The cable system of claim 1, wherein the locking ring has a first end, a second end and an engagement member extending from the second end, wherein the engagement member extends inwardly toward a longitudinal axis of the locking ring to engage an inner surface of the post.

5. (canceled)

6. The cable system of claim 1, wherein the locking ring has an irregular surface to facilitate squeezing of the locking ring from the first configuration to the second configuration.

7. The cable system of claim 1, wherein the locking ring has a circumferential wall between a first edge and a second edge, and the circumferential wall includes an inwardly extending structure permanently attaching the locking ring to a cylindrical section of the external cable.

8. (canceled)

9. The cable system of claim 1, wherein the external cable has a cylindrical section with an asymmetric cutout to mate with an asymmetric head of the post of the pedestal base, and a connector is positioned within the cutout which is attachable to a connector within a cutout in the post.

10. The cable system of claim 1, wherein the pedestal base has a first opening and a series of screw receiving openings, the screw receiving openings each dimensioned to receive a screw for attachment of the pedestal base to the skull, and the first opening is configured to receive bone chips for tissue ingrowth.

11. The cable system of claim 10, wherein the first opening is dimensioned so as not enable insertion or securement of a bone screw.

12. The cable system of claim 1, further comprising a coating on at least a portion of the pedestal base to enhance tissue ingrowth.

13. A cable system for providing power to an implantable device implanted in a patient, the cable system comprising:
a. a pedestal base mountable to a skull of a patient, the pedestal base including a post extending outwardly therefrom, the pedestal base further having an upper surface and a lower surface and first and second openings extending from the upper surface to the lower surface;
b. an internal cable electrically connectable to the implantable device; and
c. a first bone screw extending through the first opening and a second bone screw extending through the second opening, wherein the first and second screws have an initial captured position extending through the first and second openings so a distal end of the first and second screws extend distal the lower surface of the base;
d. wherein the first and second screws are in the initial captured position during shipping and are moved to a second position wherein a distal tip of the first and second screws is flush with or proximal of the lower surface for mounting of the pedestal base to the skull.

14. The cable system of claim 13, wherein the first and second screws are self-tapping screws.

15. (canceled)

16. The cable system of claim 13, further comprising an external cable connectable to the post of the pedestal base, wherein the external cable has a locking ring releasably mountable to the post, the locking ring movable from a first configuration to a second configuration, in the first configuration the locking ring locking to the post to secure the external cable to the pedestal base and in the second configuration the locking ring releasing from the post to release the external cable from the pedestal base.

17. The cable system of claim 13, wherein at least some portions of the pedestal which come into contact with tissue are coated to provide tissue ingrowth.

18. The cable system of claim 16, wherein in the first configuration the locking ring is substantially oval and in the second configuration the locking ring is substantially circular.

19. The cable system of claim 13, wherein the locking ring has a circumferential wall between a first edge and a second edge, and the circumferential wall includes an inwardly extending structure permanently attaching the locking ring to a cylindrical section of the external cable.

20. A method for providing power to an implantable device implanted in a patient, the method comprising:
a. mounting a pedestal base to a skull of a patient via application of a set of bone screws, the pedestal base having a post receiving an internal cable extending from the implantable device and tunneled from inside of a chest through a neck of the patient and to the skull; and
b. attaching an external cable to the post of the pedestal base, wherein the external cable is attached by altering a shape of a locking ring to position the locking ring over the post and releasing the locking ring to return to an initial shape to secure to the post.

21. The method of claim 20, wherein the locking ring is releasable from the post by altering the initial shape of substantially oval to a substantially round shape.

22. The method of claim 20, wherein the set of screws are self-tapping screws and the step of mounting the pedestal base comprises applying the self tapping screws through openings in the pedestal base to engage the skull.

23. The method of claim 20, wherein the set of screws have an initial captured position extending through openings in the pedestal base so distal tips of the screws extend beyond a lower surface of the pedestal base, wherein prior to mounting the pedestal base to the skull, the screws are retracted from their captured position to a second more proximal position wherein the distal tips of the screws do not extend past the lower surface for mounting of the pedestal base to the skull.

24. (canceled)
